(19) United States
(12) Patent Application Publication
(10) Pub. No.: US 2014/0222011 A1
(43) Pub. Date: Aug. 7, 2014

(54) FLEXIBLE SPRING FASTENER

(71) Applicant: Nora Systems GMBH, Weinheim (DE)

(72) Inventors: Uwe Keller, Oberhausen-Rheinhausen (DE); Gregor Grun, Zweibrucken (DE); Alfons Butscher, Birkenau (DE); Simon Garry Moore, Cambridge (NZ)

(73) Assignee: Nora Systems GMBH, Weinheim (DE)

(21) Appl. No.: 14/054,826

(22) PCT Filed: Jan. 17, 2012
(86) PCT No.: PCT/EP2012/000176

§ 371 Date: Oct. 16, 2013

(30) Foreign Application Priority Data
Feb. 23, 2011 (DE) 10 2011 012 169.2

(51) Int. Cl. A61B 17/86; A61B 17/88; F16B 19/04

(52) U.S. Cl. CPC A61B 17/8625; F16B 19/04; A61B 17/8875
USPC 606/104; 411/501

(57) ABSTRACT

The present invention relates to a connector including a body having a helix configured to change circumferentially in response to a change in force applied to the connector, the connector characterised in that the helix has at least one portion with a tapered circumference.

### PRIORITY CLAIM

This application is a continuation application of and claims the benefit of U.S. patent application Ser. No. 13/505,663 filed on May 2, 2012, which is incorporated herein in its entirety.

### TECHNICAL FIELD

The present invention relates to a novel spring fastener, a type of friction mechanism, illustrated in the form of a helical rivet.

### BACKGROUND ART

For ease of general reference both bolts and screws are understood to include a head and a threaded shaft. Bolts tend to differ from screws in that bolts tend to have an even cross-section throughout the shaft (excluding the effect of the threads) whereas screws tend to be tapered to a point at the end of the shaft distal to the head. As can be appreciated there are many thousands of variations on bolts and screws where the principles of the present invention can be applied.

Bolts and screws are somewhat impermanent in nature, whereas rivets are somewhat permanent in nature. Traditional solid rivets (a simple shaft with a head made in a deformable alloy), are not so common now, and the rivets that are generally used are “pop rivets” with an outer deformable part, (deformable by material selection and or design detailing), and a central pull element which a tool pulls on to deform the in outer part. Presently, where pop rivets are used the fastener expands to form a secure fit into the drilled hole.

It should be appreciated that the fastener industry is internationally estimated at being worth US$50 billion per annum, and the aerospace part of this is estimated to be one third.

One of the disadvantages with bolts as used presently is that it is estimated that approximately 50% of mechanical failures occur as a consequence of nuts and bolts shaking loose.

An extreme example of such failures is the crash of a Concorde at Charles De Gaulle Airport in Paris. This occurred because a small metal strip fell off a DC10 plane onto the runway.

There have been numerous attempts and many patents filed which discuss the efforts of parties around the world to invent a fastening mechanism in the form of a bolt that strongly secures elements together but does not shake loose with vibration.

Some of these mechanisms include:

In the most basic form a simple threaded bolt and nut pair are used, with very careful control of the tightening process. If the correct rotational force is applied this can be reliable, but the correct force is very hard to apply, measure or check. Common wisdom in the fastener industry is that the correct axial stretch of the bolt when best tensioned (usually by the nut) is 0.5 to 1.0% of the lineal stretch. The problem with this method is the additional time, equipment, skill and expense, required to achieve the desired forces.

Standard spring or split washers attempt to provide an axial operating force creating a bias of one thread against the other. Unfortunately the split washer is completely compressed in use, and therefore largely acts as a standard flat washer, with a small anti rotation benefit only if the leading edges of the split area are sharp.

Loctite™ is anaerobic glue which can be effective in binding threads but is very sensitive to cleanliness and temperature, somewhat messy to use, requires a close tolerance between the cooperating elements

Using a pair of threaded nuts is a strategy used sometimes. This improves the vibration resistance but is cumbersome slow and adds expense. Additionally it only improves vibration resistance a little as the axial stretch of the fastener still determines the efficacy of the thread friction engagement (now on two nuts).

Castle nuts are used where the shank of the bolt is pierced, and a pin is able to pass through both a pair of castellations on the nut, and the hole in the bolt shank, thereby avoiding rotation or the castle nut. This improves the vibration resistance but is cumbersome slow and adds expense.

Patent number DE 10204721 discloses a spring bolt which enables the length of the fastener to change. This has a helical spring which extends from the head of the bolt and connects to a solid threaded region. This only has a small threaded portion at the end thereof joined to the non threaded flexible spring. This device does not have the strength of even conventional bolts.

Patent number JP 2005/325,999 discloses a fastening mechanism a first fastening member having a vibration source to a second fastening member. This is a means to dampen vibrations, not to provide a strong secure fastening. Again it only has a small threaded portion which is attached to a spring.

Patent number PCT/IL2001/00924 discloses an interested fastening mechanism which has a variable pitch thread configuration. This consists of a split threaded cylinder which in its resting states has threads substantially parallel to each other.

Twisting the cylinder in the appropriate direction creates either a right hand or a left hand thread. The cylinder is not fixed to a head as such and as a consequence of the split along its length provides a flexible, but not very strong fastening device.

U.S. Pat. No. 4,917,554 discloses a corkscrew like fastener used to join together semi-rigid mats. This consists of a head and shaft wound from circular wire in the form of a helix. While this is useful with amorphous products such as mats, this can not be used where structural strength is required. The round wire of the ‘corkscrew’ cannot readily cooperate with a solid object nor provide the strength, grip or fine tolerances that a simple threaded bolt can.

To avoid vibration loosening of threaded fasteners, and shear failure, often designers specify the use of rivets. For example in a Boeing 747 “Jumbo” plane there are about 4 million fasteners and many of these are rivets.

There are several significant problems in that the cost of the fasteners and the cost of installation is a significant cost in making a new plane and then the plane needs to be able to be inspected and maintained in a working life of often in excess of 30 years.

Indeed for maintenance and inspection of plane substructures, the relatively permanent nature of rivets is a problem, as pop rivets as available now require tedious and costly drilling out, and then the reinsertion of a new pop rivet. It would be much better if a rivet were very secure but able to be removed quickly and even reused as an option.

Whilst fastenings can be visualised more conventionally as a nut and bolt holding together planes, cars, bicycles, toys, furniture, and machinery, they are present in a myriad of applications.

Where ever fasteners are used the same challenges are present: Fasteners usually need to be affordable, easy to insert/use, secure, vibration resistant, corrosion resistant, and often able to be removed if required.
However there are also less obvious “fasteners” that are actually connection details/sub-parts of a larger item. Some examples of connection details/sub-parts are cams, dovetails, wedges, threads, levers, snap-fits, zips, press-fits, friction-fits, and interlocking details.

The prior art illustrates a myriad of forms to connect the screwdriver bit to the head of the fastener. However there are still a number of problems:
1. The bit is prone to torque out—where the drive bit disengages from the fastener recess, as the power is applied,
2. The bit does not hold the fastener and the fastener falls out of the bit before it can be used,
3. The recess in the fastener is easily compromised by paint and/or corrosion plating,
4. The recess in the fastener is expensive to form.

An ideal fastener-bit interface would address all the above problems, and preferably be more secure as the power is applied. Ideally the fastener-bit connection would be more secure as the power increases.

Yet another fastener problem is in the area of pop-rivets which are slow to insert, relatively expensive, and can not be removed easily, or reused. This invention will address these problems with a novel form of “spring rivet”.

Other than conventional applications noted above there are also less obvious needs for fastening solutions such as in medical and dental applications, where reconstructive repair to bones or the teeth often require the use of fastenings.

Bio compatible metal alloys (or composites) can be used for pins, screws, plates, ball joints/sockets, implants, and the like, but there are un-solved issues with the prior art. These relate to the relative inflexibility of the parts, and the challenges fitting to the existing organic bone structure:
1. Materials such as the titanium alloys used in orthopaedic and dental reconstructions are particularly strong, but therefore relatively inflexible. It would be better it these parts could be designed to be able to flex under sever load to avoid failure of the medical device to bone interface, particularly before adjacent bone “knits” into the part.
2. Biological features are never simple geometric forms, such as cylinders or cones. For example the cavities within bones are far from regular in course, form and cross section. Specifically when a tooth is removed the cavity which remains in the jaw bone is somewhat organic and tapered in form and varies significantly from person to person, and indeed tooth to tooth. Conventionally the jaw is drilled out to a regular form, so that a rigid form fastener element can be threadably engaged. It would be safer, cheaper and quicker to have a connection solution which can be engaged securely to the organic and varied forms of the cavity as nature formed it, or with minor adjustment. This would leave intact more bone, preserve the character of the established matrix, and also avoid delay in waiting for the bone to re-grow before the drilling occurs prior to implantation
3. Human skeletal structure is not static. A connection system to the human form would be improved if it could not only fit to the form as it is, but was also capable of adaptation, or adjustment, particularly for growing children, or alternatively capable of removal.

Dental implants are relatively common procedures and serve as a good example of a connection technology in the biomedical area with unresolved challenges: A dental implant is an artificial tooth root replacement and is used in prosthetic dentistry to support restorations that resemble a tooth or group of teeth. In its most basic form the placement of an osseointegrated implant requires a preparation into the bone using either hand osteotomes or precision drills with highly regulated speed to prevent burning or pressure necrosis of the bone. After a variable amount of time, to allow the bone to grow onto the surface of the implant (osseointegration), a tooth or teeth can be placed on the implant. The amount of time required to place an implant will vary depending on the experience of the practitioner, the quality and quantity of the bone and the difficulty of the individual situation. Failure rates of about 5% are quoted, mainly due to failure of osseointegration. But there is a significant problem in the delay in completing the procedure.
An increasingly common strategy to preserve bone and reduce treatment times includes the placement of a dental implant into a recent extraction site. In addition, immediate loading is becoming more common as success rates for this procedure are now acceptable. This can cut months off the treatment time and in some cases a prosthetic tooth can be attached to the implants at the same time as the surgery to place the dental implants. However, the chances of the implant failing in these cases is increased.
When an implant is placed either a healing abutment, which comes through the mucosa, is placed or a ‘cover screw’ which is flush with the surface of the dental implant is placed. When a cover screw is placed the mucosa covers the implant while it integrates then a second surgery is completed to place the healing abutment.
Two-stage surgery is sometimes chosen when a concurrent bone graft is placed or surgery on the mucosa may be required for aesthetic reasons. Some implants are one piece so that no healing abutment is required.
In carefully selected cases patients can be implanted and restored in a single surgery, in a procedure labelled “Immediate Loading”. In such cases a provisional prosthetic tooth or crown is shaped to avoid the force of the bite transferring to the implant while it integrates with the bone.

Surgical timing: There are different approaches to place dental implants after tooth extraction. The approaches are:
1. Immediate post-extraction implant placement.
2. Delayed immediate post-extraction implant placement (2 weeks to 3 months after extraction).
3. Late implantation (3 months or more after tooth extraction).

According to the timing of loading of dental implants, the procedure of loading could be classified into:
1. Immediate loading procedure.
2. Early loading (1 week to 12 weeks).
3. Delayed loading (over 3 months)

Generally the above prior art dental implant processes are overly dependent/reliant upon both the speed and success of osseointergration process. It would be better if the implant were able to mechanically connect immediately, and very securely, to the recess, and preferably without drilling the jaw, and then over time the osseointergration process would serve to make the mechanical connection additionally secure, and permanent.

At edentulous (without teeth) jaw sites, a pilot hole is bored into the recipient bone, taking care to avoid the vital structures (in particular the inferior alveolar nerve or IAN and the mental foramen within the mandible).
Drilling into jawbone usually occurs in several separate steps. The pilot hole is expanded by using progressively wider drills (typically between three and seven successive drilling steps, depending on implant width and length).
Care is taken not to damage the osteoblast or bone cells by overheating. A cooling saline or water spray keeps the temperature of the bone to below 47 degrees Celsius (approximately 117 degrees Fahrenheit).
The implant screw can be self-tapping, and is screwed into place at a precise torque so as not to overload the surrounding bone (overloaded bone can die, a condition called osteonecrosis, which may lead to failure of the implant to fully integrate or bond with the jawbone).
Typically in most implant systems, the osteotomy or drilled hole is about 1 mm deeper than the implant being placed, due to the shape of the drill tip. Surgeons must take the added length into consideration when drilling in the vicinity of vital structures.

The time and risks in drilling or modifying the jaw bone, can be reduced somewhat by the use of CT scanning: When computed tomography, also called cone beam computed tomography or CBCT (3D X-ray imaging) is used preoperatively to accurately pinpoint vital structures, the zone of safety may be reduced to 1 mm through the use of computer-aided design and production of a surgical drilling and angulation guide. However despite this it would still be safer, and quicker, if a connection device could be fitted to the jaw aperture as found or with minimal jaw modification, and that a limited inventory of devices could be readily available to be used.

All references, including any patents or patent applications cited in this specification are hereby incorporated by reference. No admission is made that any reference constitutes prior art. The discussion of the references states what their authors assert, and the applicants reserve the right to challenge the accuracy and pertinence of the cited documents. It will be clearly understood that, although a number of prior art publications are referred to herein; this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE AND BEST MODES OF THE INVENTION

According to one aspect of the present invention there is provided a connector including

a body having a helix configured to change circumferentially in response to a change in force applied to the connector,
the connector characterised in that
the helix has at least one portion with a tapered circumference.

According to an alternate aspect of the present invention there is provided a driver bit for the application of rotational torque to a connector as described above,

characterised in that
the driver bit includes a body having an attachment portion at one end capable of connection to a rotational driver, and
the body having at the distal end to the attachment portion a shaft configured to engage with the internal circumference of the connector.

According to a further aspect of the present invention a method of fastening a connector as described above,

the method characterised by the steps of
a) inserting the connector into a component aperture
b) applying rotational torque via a driver bit described above to the connector to secure the connector within the aperture
c) applying further rotational torque to release the driver bit from the connector.

According to yet another aspect of the present invention there is provided a fastening system including a connector and a driver bit as described above. Some versions of the fastening system may also include components with pre-drilled apertures to receive the connectors.

Is should be appreciated that the force that causes circumferential change can come in a number of forms.

In one embodiment, the force is linear in application and therefore the circumference of connector changes as a result of either lineal tension being applied to the body, or compressive forces being applied.

However, in preferred embodiments of the present invention the force applied to the body to change its circumference is in the form of rotational torque.

It should also be appreciated that it is a change in force that causes circumferential change. Thus, application of rotational torque may cause the circumference of the connector to increase or decrease. Likewise the release of rotational forces on the connector can cause a circumferential change as well.

It is envisaged that the tapered circumference could be the external circumference of the connector or the internal circumference, or in some embodiments both. The importance of the taper will be discussed in detail later on in the specification, but a brief overview of its usefulness is given below.

A connector with a tapered outer circumference allows the connector to be pushed partially into an aperture to form a frictional fit. This engagement of the connector with the aperture is important as it holds the connector in place at the start of the application of (or release) of the force applied to the connector to change its circumference.

For example, the connector may be pushed partially into a hole until it cannot go forward any further. Then, rotational torque can be applied to the connector which causes its outer circumference to decrease. This allows the connector to be then pushed further in to the aperture. Upon release (or application) of rotational torque, the outer circumference of the connector will increase thus forming a very strong fit within the aperture.

Likewise, a tapered internal circumference of the connector can provide a frictional fit.

In preferred embodiments, this frictional fit is with a driver bit that applies rotational torque to the connector. For example, the driver bit may have at least a partially tapered shaft which can be positioned within the connector until it grips on the internal surfaces of the connector. The application of rotational torque to the driver bit translates through to the connector as a consequence of that frictional fit.

Some embodiments of the present invention continued application of rotational torque causes the driver bit to engage and disengage with the connector.

It has been recognised by the inventor that the principles of the present invention can also be applied to the use of a connector with substantially parallel internal and external sides in combination with a tapered aperture. Trials conducted by the inventor has shown that this means of providing frictional engagement is not as successful as with the preferred embodiments of the present invention. However, it is recognised by the inventor that this is a potential embodiment of the present invention and that in some embodiments there may be provided a fastening system with pre-drilled tapered holes and parallel sided connectors.

It should be appreciated that it is envisaged that the material from which this connector may be made is preferably of a type, and construction, that possess a material “memory”. This means that if the rivet is deformed through forces placed on it, there is a natural tendency for the material “memory” to bias the rivet back towards its original shape.

### DEFINITIONS

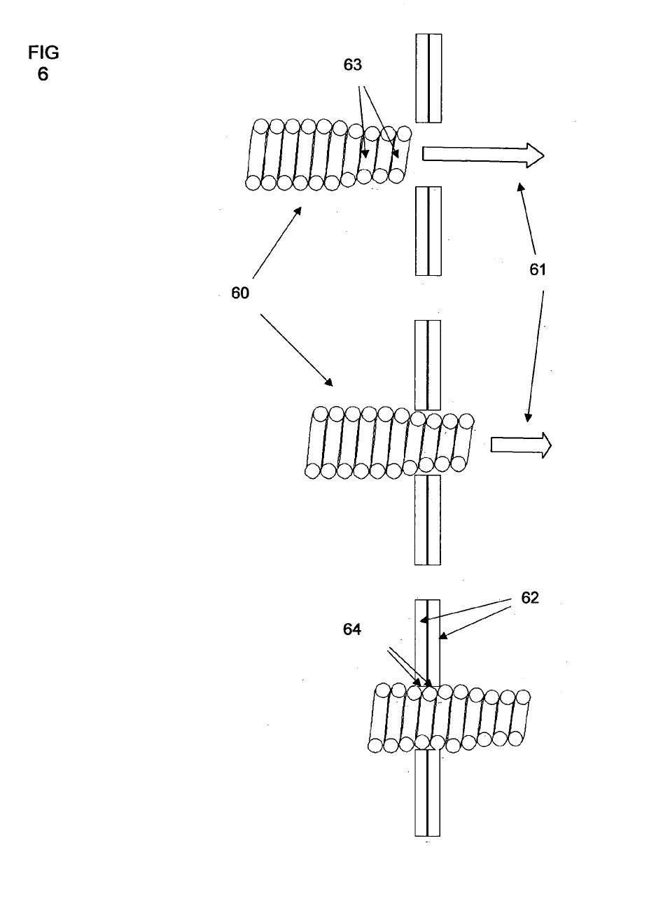

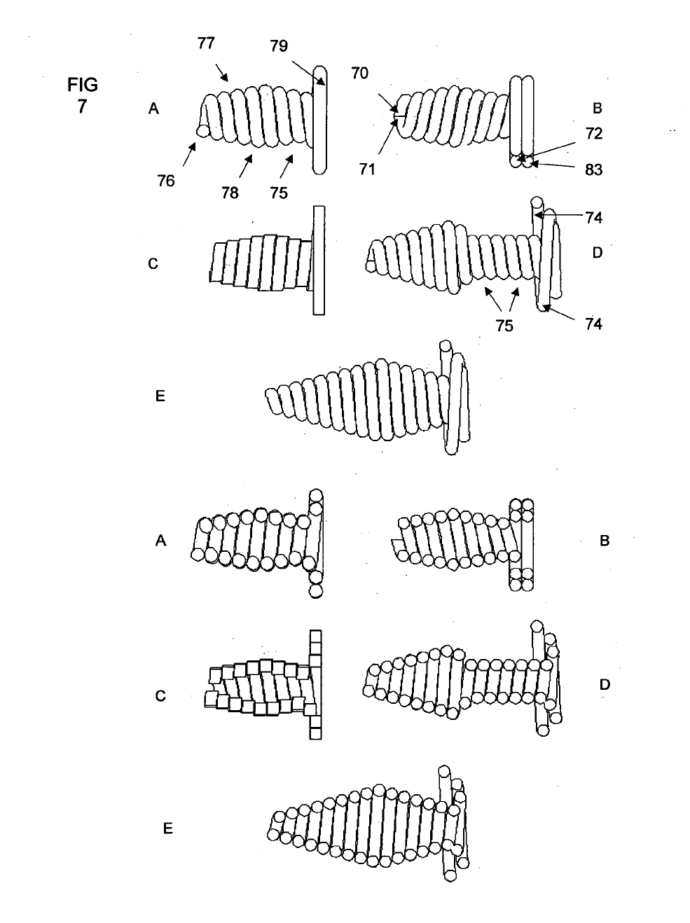

For the purposes of this patent disclosure the following definitions apply, and are integral parts of this invention disclosure:
1. The term “helical” means either “spiral or helical” in the normal literal sense, but also can be taken to mean “capable of radial expansion and contraction, with or without associated lineal contraction and expansion”. A helix is generally a shape capable of simple mathematical description via specifying its length, diameter, and pitch.
   a. The cross section is generally, but need not be, constant. The exterior diameter may be constant, but need not be. The interior bore diameter may be constant, but need not be. The pitch is generally, but need not be, constant.
   b. An example of more unusual helices under this definition are hollow woven braided tubes, and elastic material tubes.
   c. Whilst the cross sections of the drawings herein are generally square, and otherwise round, this is not limiting in any way, and any cross section may be used for iterations to advantage, including rectangular, oval, irregular, and modified round (perhaps ground).
      i. The cross section could be defined to be custom to a particular fitting requirement. For example after a CT scan—or similar—of the jaw or bone, the artificial part could be custom designed so it can helically engage to, or otherwise fit to, the organic form of the jaw or bone. This could lead to the form of the helical insert being quite different in situ than originally made.
2. Helical can therefore mean all of the following:
   a. A tube with an elastic wall,
   b. A corrugated (perhaps helical) wall tube,
   c. A tube with one or more helical slots, or a tube form with one or more helical slots,
   d. A tube with one or more generally linear slots,
   e. A single simple corkscrew like detail,
   f. A helix which is in the nature of a standard extension spring—with the winds actually touching each other—or alternatively the helix may semi extended, which is shown in the drawings where a slight gap can be seen between the winding helix forms,
   g. Several corkscrew details, which has the clear advantage of better resisting a single corkscrew detail being inadvertently twisted off a wire with sideways force application.
   h. An extension or compression spring,
   i. A multi start helical design,
   j. More than one helical detail adjacent to each other (including where there is one helical form overlying another non helical or axial detail),
   k. A woven bidirectional helical design in the general character of a braided tow rope, or a Chinese finger trap/pull toy.
   l. A bidirectional helical design in the general character of overlapping helical details which are clockwise and anti clockwise.
   m. A spiral form where there is considerable overlap of overlying layers of material (FIG. 6e)
   n. A helical form where the helical angle is shallow or “slow” as in normal springs, say 1-10 degrees, or with any other steep or “fast” wind, for example 80 degrees. The later can be visualised via a simple form with a number of generally co-axial, rods with a central common axis, which gently wind about the axis (FIG. 7).
      i. A helical form with a variable pitch where it is slow in area and fast in other areas.
3. The term “fastener” shall include a temporary, permanent, adjustable, and fixed fastener and any part or subpart which serves to fasten the larger whole part.
4. The term “mechanism” shall include any device, assembly, or unitary item.
5. “A bore aperture” bore aperture, is any internal wall capable of receiving a fastener. It would include for example: one or more sheets or plates with a hole; a housing; a cast body; a nut (threaded or plain), and a natural modified or formed cavity in a bone or other body part. A bore aperture may have a constant internal diameter, or may be tapered in an area. The proximal end for receiving the fastener may have a lead in area of a slightly larger internal bore diameter.
6. The connector of the present invention shall for ease of reference be called a spring fastener—although this should not be seen as limited. The term “spring fastener”, shall be taken in the broadest sense including the use of simple one start helix such as a simple spring, compressed springs, extended springs, helical mechanisms, complex multi-spring assemblies, two start springs with 2 or more adjacent winds made from a single piece of metal/plastic/polymer which winds back and forth, multi-start helices, overlapping helical element, threadably co-operating helical elements (where one part is threadably inserted or wound at least partially into another), braided or interwoven parts (including soft or spring forms of a braided rope or Chinese finger trap), and other parts which because of their design, and/or materials (perhaps elastic), can act as springs in use. Any adjacent part or material may be considered as either a separate part or as an integral part of a spring fastener.
   a. For the purposes of this patent a useful visualisation of a spring fastener is a single (one start) helix of tightly-wound and heavy square-section spring-wire, or a “simple square spring rivet”, with a distal end which has a slight taper for a lead in, a central area which will lock to the bore aperture in use, and a proximal end with a central bore for attaching a drive part such as an electric screwdriver bit.
      i. Whilst an simple square spring rivet may have a defined and visible head detail at the proximal end this is not necessarily required, as the head may be effectively formed by an uncompressed part of the simple square spring rivet when in use. The part of the simple square spring rivet not inserted into the bore aperture will retain its original diameter and act as a head.
      ii. Likewise the simple square spring rivet may not require a nut at the distal end as the distal part of the simple square spring rivet inserted through the bore aperture will “elastically regain” its original diameter and act as a nut.
      iii. Therefore a simple square spring rivet effectively forms both its nut and head by the constriction radially of the central area. Further the compression of the central area will lengthen this area and there will be created a resultant force that will pull the “head” and “nut” ends towards each other. This will help secure the parts, and being elastic will naturally resist vibration.
7. The “internal bore” of a spring fastener shall be the innermost surface, which would be very lightly touched by an inserted rod just capable of insertion, i.e. without changing the length of diameter of the spring.
8. The “external surface”, of a spring fastener shall be the outermost surface.
   a. In a bolt, nut, or screw the external surface is generally threaded.
   b. In a rivet the external surface is generally non-threaded or plain.
   c. Generally the external surface is illustrated as plain in this description, but this is for simplicity of illustration only, as the external surface may be threaded, barbed, textured, or irregular.
9. The shaft of a fastener is that part which is between the head and nut end, and confers the main structural strength. Generally in a spring fastener the helix itself is the functional shaft.
10. A “self locking device” or “self locking mechanism”, is a part which substantially defines a locking force by itself, so the action of a person is to assemble the parts, but the primary locking force can be considered to be less dependant on the persons skill, intent, or strength, but more determined by the mechanical and design character of at least one part.
11. An “interference fit” is a frictional engagement between touching adjacent parts, where adjacent surfaces may have features which are smooth/textured, rigid/elastic, parallel/tapered, circular/non circular cross-section, regular/irregular cross-section, ribbed/plain/splined, with matching/non-matching taper angles, or any other arrangement/combination.
12. The terms “wires, rods, tubes, cables, or other elements which have at least one generally linear aspect” shall be interchangeable.
13. Generically, a thread in the present invention is a spiral ridge extending along a surface, wherein the threads themselves are helical in form. In preferred embodiments the threads are of a fairly conventional form with a sharp or tapered edge, which can readily cooperate with complimentary threads in the same means as a conventional bolt and nut. It is this interaction that gives requisite strength, grip, fine tolerances and required interaction between the two objects.
14. A super elastic material is defined as one which may deform in a manner where a dimension can increase by a factor of at least 1.5 times, but then subsequently is capable of elastically returning to the original dimension.

According to an embodiment of the present invention there is provided a spring fastener, wherein at least part of the shaft is effectively helical, and at least part of the shaft has no anchoring base, characterised in that the shaft is formed from at least one unbroken wound spiral. The spring fastener is by design capable in use of dimension changes including axial stretch (length change), and radial reduction (diameter change), and vice versa.

According to an embodiment of the present invention there is provided a spring fastener which is by design, and material selection, capable—in use—of dimension changes including axial variation (length change), and radial variation (diameter change). Commonly when the spring fastener is used the length will increase and there will be an associated area of local radial reduction.

According to an embodiment of the present invention there is provided a spring fastener in the nature of a modified simple square spring rivet, where there is a defined head detail visible before use. This head may be solid in form or with a helical slot.

According to an embodiment of the present invention there is provided a spring fastener in the nature of a modified simple square spring rivet, where there is a defined nut detail visible before use.

According to an embodiment of the present invention there is provided a spring fastener, where there is an integral elastic material, perhaps natural or synthetic rubber/elastomer. This elastic material can prevent the passage of liquid or gas. This elastic material may be injection molded or an insert part which assembled into the spring fastener.

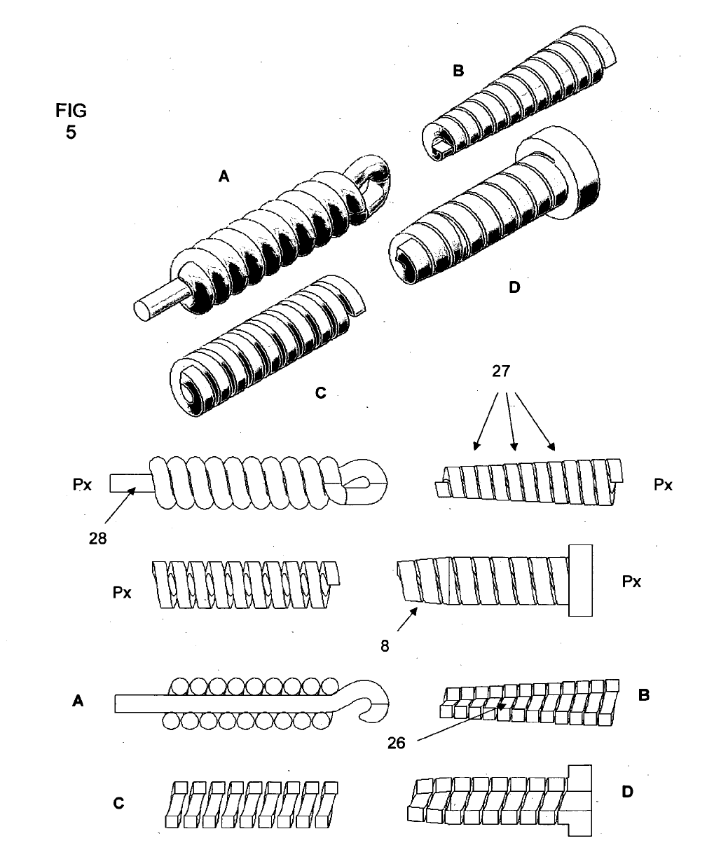

According to an embodiment of the present invention there is provided a spring fastener, which pre-stretched and/or pre-turned prior to insertion and use, or is mechanically (forcibly) stretched in length and/or rotated as it is inserted into a bore aperture. The insertion may be generally linear—coaxial to the bore aperture bore axis—and/or by winding the spring fastener into the bore aperture.
The stretch may be gradual, and in winding in, or more sudden as in hammering.
There may be an internal stretch enabling element, or pre-turn enabling element, which can be a pin attached or a separate threaded element.
An internal element may be continuous, with a helical form, as in FIG. 5a. Here the end detail may be hit, pushed or grasped, and turned to insert the spring fastener. The spring fastener may have a lead, or the bore aperture may have a lead. Any internal drive pin or element may be designed to be retained, or removed (in which case it may include a pre defined snap-off point or weaken area.)
An advantage of such an arrangement is that by accessing the helix at the distal end both the insertion actions of stretch and turn, may first occur at the distal end which first has to be inserted into the bore aperture. The distal end can be smaller in an outside diameter than an internal dimension of a bore aperture and therefore be inserted into the bore aperture and thereafter a tool engaged to the distal end can be turned to progress the spring fastener further into the bore aperture.
Alternatively an internal element could itself also be helical in form, and this may aid removal of a spring fastener.

This invention also describes an insertion tool with a general end pin detail, perhaps with a shoulder which prevents the tool extending into the spring fastener excessively (where it may expand the spring fastener too much, so that a proximal part is not capable of full insertion into the bore aperture.
The tool end detail may be tapered or parallel.
   The tool end detail may be configured to be parallel over a length and then tapered towards the tip that first engages with the distal end of a simple SR
   The tool may have a hex detail at one end in the naure of screwdriver bits commonly available. In this example the cross section of the tool would be in three parts:
      1. Generally hex in form
      2. Generally parallel in form
      Generally circular and tapered in form
The tool end detail may be asymmetrically detailed so that as it is turned in one direction particularly it holds/grips well so that the spring fastener may be either pushed or pulled. This is advantageous as a feature of spring fastener is that the direction of the turn to insert is the same as that to remove. Also many spring fastener forms will not have an external conventional thread (although they may be helical in structure), and as such the normal “right is tight” to insert and “left is loose” sequence is not possible, and specifically there is no thread to axially progress the fastener differentially depending on the rotation direction.
   An example of an asymmetric detail for this purpose is a square end detail which is slightly spiral in form, which can fit into a similarly shaped aperture in a solid fastener proximal end. (This asymmetric detail can be visualised as a standard square drive bit—slightly twisted at one end)

If a spring fastener is “pre-stretched” and/or “pre-turned” there may be a part which is removed, (once the spring fastener is correctly positioned in the bore aperture), thereby letting the spring fastener regain its original shape, and lock into the bore aperture, (forming a head and nut detail if so detailed/desired). To stretch or turn a spring fastener will often require a generally distal detail, perhaps solid, or a retained inserted part, to push and/or turn against. The push/or turn action can be as the spring fastener is used or before the spring fastener is used, where it is pretensioned linearly and/or helically, before use and assembly.

Generally a spring fastener of any form including a simple square spring rivet or solid drive bit needs to be attached temporarily or permanently to a first adjacent/attached object, and also a second adjacent/attached object.

For example:
In the case of a simple square spring rivet the first attached object could be a driver bit in the form of a solid drive bit, perhaps tapered, and the second attached object could be a bore aperture

In the case of a solid drive bit the first attached object could be a chuck of an electric power tool, and the second could be a bore aperture in a screw or an external surface of a screw/bolt/pin/fastener. According to an embodiment of the present invention there is provided a spring fastener, which is capable of being compressed and/or turned after insertion, thereby shortening the helix and expanding it (forcibly) against the bore aperture. The compression could be via an inserted element, such as a threaded part.

According to an embodiment of the present invention there is provided a spring fastener, which utilizes a super elastic material.

According to an embodiment of the present invention there is provided a spring fastener, which utilizes “super metal memory” material (which is capable of alternate crystalline structures—with dimensional change) so that the spring fastener can be inserted and then by the application of heat or cold its shape can be changed to either secure or remove the spring fastener. Nickel titanium alloys are an example of such materials.
The use of these metals in spring fastener is perhaps an extreme example of self locking mechanism design.
Of course the application of heat or cold could also be used with more common materials to insert, secure or remove an spring fastener.

It is envisaged that the principles behind the fastening mechanism of the present invention can be used in a variety of situations. For ease of reference however the fastening mechanism shall be referred to as a rivet, often a simple square spring rivet. It should be appreciated however that this is not intending to be limiting.

Also, it should be appreciated that the present invention could cooperate with complementary threads (such as in a nut) or directly into a material.

The head of the present invention can be of any shape or configuration required for the spring fastener to be “done up” or “undone”. For example, the head may be hexagonal with sides of a shape and size designed to cooperate with standard spanners and the like. In other embodiments, the head may be designed to cooperate with various screw drivers, such as chisel or flat head or Philips head. In other embodiments the head may have a recess which is designed to cooperate with the end of an Allen key, shaft, or tapered shaft.

The shaft likewise can be any length or thickness suitable for the particular application in which it is intended to be used.

With the present invention, the general form of the spring fastener is not with a solid internal shaft as with the prior art. The inventor has deduced that the solid shaft in the prior art acts as an anchoring base which gives this inflexibility of movement.

It should be noted that in a general form of this invention the structure is formed as one from at least one unbroken round spiral. This means that there is at least a 360° turn to form the shaft and thread. Naturally in preferred embodiments there are many such turns.

Generally at least a part a spring fastener will have an outer diameter greater than at least part of the inner diameter of a bore aperture to which it is to fit to. The action of screwing the spring fastener into the bore aperture can cause the spring fastener to compress (and/or lengthen) under the pressure of this action. This is possible because there is no central core to resist the compression. However, once the screwing action has stopped, the natural memory of the material from which the “shaft” is made (in combination with the spiral form) causes the spring fastener to extend outwards in an attempt to resume its original shape. It is this action that causes the external surface of the spring fastener to form an interference fit with the bore aperture.

Likewise, to remove the spring fastener from the bore aperture requires a screwing action which again will cause the spring fastener to compress (and/or lengthen), making it easy to remove. Surprisingly the direction of removal of a spring fastener is the same as for insertion. So whereas a normal thread is “right is tight” and “left is loose”, a spring fastener is either “right is tight and loose”, OR “left is tight, and loose”.

An advantage of the present invention is simplicity, and indeed in its simplest form the tool bit which drives the simple square spring rivet into the adjacent bore aperture need not even be square, slotted, hex Phillips™ or Posidrive™, as it need be no more than a simple circular cross section pin or shaft element—perhaps a reversed drill bit, drill blank, or tapered round cross section drive part.

In use the following sequence can occur:
1. The drive part is fitted into the distal end of the simple square spring rivet by (say) anti-clockwise rotation, or a push fit. This can be conveniently achieved by having the drive part in an powered screwdriver
2. The simple square spring rivet is pressed to the bore aperture (to which it is oversize), where in a preferred form either or both the simple square spring rivet/bore aperture have a taper/lead to facilitate initial engagement of the two parts. The taper/lead for the simple square spring rivet would be in the form of reducing the outside diameter, whereas the taper/lead for the bore aperture would be in the form of increasing internal diameter,
3. When the power tool turns the drive part clockwise, the simple square spring rivet is also turned clockwise.
4. The insertion of the simple square spring rivet into the bore aperture is eased when the lead of the simple square spring rivet begins to grip the bore aperture, as there is momentary resistance which causes the following effects simultaneously:
   a. The drive part frictionally engages more securely to inside of the simple square spring rivet helix, by tightening helically to the proximal end, and
   b. The simple square spring rivet begins to reduce in diameter.
      (If the cross section of the helix is constant the reduction in diameter will be uniform along the length of the simple square spring rivet, other than where there is a diameter, pitch, or other variation)
      Note: The above sequence uses the observation that a first solid part wound into the inside of a simple spring (say clockwise) will grip very securely, but a second solid part wound in the same direction, (also clockwise) over the same end of the very same spring does not grip at all, and actually it needs to be turned the opposite direction (anti-clockwise) to grip.
5. As force is applied axially and the drive part continues to turn the simple square spring rivet which can be progressed into, and through the bore aperture (if desired).

The tool bit described above is a solid drive bit, solid in form, and perhaps tapered, but alternatively the tool bit itself may be a helical form, to be used with an simple square spring rivet, and this invention also describes the use of a helical tool bit used in any aperture, for example a bore aperture such as a parallel, tapered, or dovetailed circular or oval hole in the head of a solid fastener.
a. The helical tool bit would be of any suitable cross section, and may be tapered or parallel.
b. A helical tool bit is capable of securing to the screw only in one direction of rotation so there could be a helical form on the other end of the bit with the opposite rotation of its helix.
c. A helical tool bit fitted to a bore aperture could be used for any number of tools including screwdrivers, assembly robots, lathes, milling machines, routers.
d. A helical tube bit may have a single start helix or a multi-start configuration, and may have a central aperture, but it may also be in a solid helix form, without a central aperture, which can be visualised as an auger form which is axially shortened. A flat wrap form is possible where one end is in then nature of overlapping layers as in FIG. 6e.
e. The exterior surface which engaged to the bore aperture or screw may be polished, threaded, textured, knurled, or finished in any way.
f. The helical tool bit could have a solid core and at least one flexible outer helical part. In use the outer part may be forced onto the inner core part.
g. The helical tool could be either a unitary item or made of sub parts, for example:
   i. A simple spring element circular or other cross section, perhaps a heavy square section closely wound spring (maybe tapered),
   ii. A spring element circular or other cross section retained within an outer structure, perhaps tubular.
   iii. A simple spring element circular or other cross section retained within an outer structure, perhaps tubular, where the simple spring element is forcibly wound into the outer structure,
   iv. A simple spring element circular or other cross section retained within an outer structure, perhaps tubular, where the simple spring element is retained within the tubular element by an inner most third part, or by welding gluing or other means.
h. A helical tool bit has been described above in a male form, but the principle is flexible and could be configured to be in a female form, easily visualised as a general heavy wall tube form with a helical slot.

It should be appreciated that in alternative embodiments a spring fastener may be partially, in the form of the current invention and partially in the form of the prior art. Therefore this invention describes a hybrid fastener, with self locking detail, which for example may be partially a conventional fastener with a solid core, and partly a fastener which is without a solid core, or any other detail as described in this specification.

Where a spring fastener has an external thread, the torsional forces from the screwing action cause the pitch and thread spacing of the shaft to alter and conform to the complementary thread with which it is being used.
The torsional forces from the screwing action cause the pitch and thread spacing of the shaft to alter and conform to the complementary thread with which it is being used. If the pitches and or thread detail are different then relative rotation will cause friction which leads to elastic deformation of one or both parts. This will apply particularly to the externally threaded part as it has no solid core at least part way in its axial length, and as such is better able to elastically deform (lengthen or shorten depending on the details of the two parts).
Generally in the embodiments herein described for at least part of the fastener axial length the pitch detail differs form the pitch detail of the cooperating part and this creates a controlled mismatch of the parts. When the two parts engage there is a requirement that one or both parts compress or stretch to align the threads and allow continued rotation.
In comparison it has been found that a conventional spring will not work as a self locking spring thread fastener if its thread is in frictional resistance with the female thread, as the simple action of turning the fastener thread causes the spring helix to unwind and become larger in diameter, thereby stopping further progress.
The present invention has a structural thread, and is thread capable of resisting expansion in diameter but accommodating the pitch mismatch by axial stretching.
In comparison a simple spring detail will bind and be able to rotate no more. The structural thread is the non solid helical core
Thus it can be appreciated that the torsional action of screwing and unscrewing the bolt compresses the shaft providing a product which is easy to undo or do up. However, any vibrational forces subsequently resulting are easily resisted by the bolt and nut combination as a consequence of the memory of the shaft pushing the threads out against the internal threads of the nut.

It should be appreciated that a bore aperture may be in fact a nut in function.

Means by which such a spring fastener can be formed are varied, including for example: helical extruding; injection molding; lost wax casting, roll formed, pressure formed, stamped, die cast, sintered, additively printed, machining, removal of stock or any other method.

All helical form spring fastener may be single helix, or a multiple start helix in form.

The present invention can be beneficially made with an internal bore diameter which is similar or less than the diameter of the helical form wire cross section. So if the wire is say 6 mm in cross section, the internal bore may be 6 mm or thereabouts in diameter.

The present invention can be beneficially made with an internal bore diameter which is substantially less than the diameter of the helical form wire cross section. So if the wire is say 6 mm in cross section, the internal bore may be 1 mm or thereabouts in diameter.

In some embodiments of spring fastener, a central aperture could be filled with a buffering or lubricating material.

In some embodiments, the aperture could serve as a passageway for substances to pass through or be a means by which a further attachment can be connected to the bolt.

One means by which a spring fastener can be put in place, other than the afore-mentioned pre-stretch, is to effectively compress the spring fastener prior to or during insertion into the material, so the act of compression causes the external diameter of the spring fastener to decrease temporarily. Subsequently, after removal of the compression force, the spring fastener will expand to the bore aperture, and be secure.

Most embodiments of this invention are is in the form of a self locking mechanism, and specifically a self locking rivet, but the locking force may be solely by or augmented by another part, or the application of energy, such as heat.

A spring fastener can be envisaged as being made from a long strip of material which has been wrapped in spiral form with the edges of the strip forming threads or barbs.

A spring fastener can come in parallel (as in a bolt or solid rivet in the prior art), or tapered form (as in a wood screw in the prior art).

It can be seen that the present invention and all of its embodiments provides significant advantages over prior art.

It should also be apparent that the simplicity of design of the present invention means that the fastener mechanisms can be relatively easily manufactured using known techniques.

It can also be seen that the present invention can be provided in the form of a kitset including a spring fastener and bore aperture designed to work as a pair.

This invention is described here by way of fasteners, and specifically a rivet, but equally the principles of the invention herein can be applied directly to any number of non-fastener items to be connected, such as for example: machinery, sports equipment, scaffolding, tube connection, furniture, toys, and any application where parts need to be secured together temporarily or permanently.

To enhance connection in bore aperture which are tapered, somewhat organic, or irregular, such as a hole or fissure in rock, or a the there will be some advantage in preparing the bore aperture so that there is a pre formed reverse taper or ridge recess detail. This invention describes a novel asymmetric mill cutter that has a central shaft and an asymmetric cutting detail at one end which extends farther from the cutter mill axis on one point than another side opposite. In use this cutter may be for example cut a groove or dovetail recess in the bottom of a cavity in the jaw bone after tooth extraction.

In situations where there is a danger of milling or drilling too deep, as in dental implant procedure, it would be advantageous to use a drill or a mill which is unable to cut at the base, or has a shoulder or depth stop to limit the depth of cut. The use of a taper drill or taper mill, with or without a shoulder, would enable the surgeon to easily ascertain the depth of cut. A tapering tap could then be used if desired and a helical insert or a solid insert then wound in.
The bore aperture modification could be partial or full depth, so that if partial the proximal part of the irregular organic cavity could be formed to a more convenient regular shape/cone and the distal part left unaltered.

Any fastener helical form described in this invention could be defined as a plug for receiving an internally located part such as a pin, rod, wedge, taper, threaded element, second helical form, second fastener, buffer, plug, elastomer, or other expansion or restriction part.
The internally located part could itself receive a third part—internally located to itself.

A general form which has many applications is an expansible helical plug which, after insertion to a bore aperture, is made incapable of constriction by the presence of the internal restriction part.

### BEST MODES FOR CARRYING OUT THE INVENTION

Further aspects of the present invention will become apparent from the examples in the accompanying drawings, which are integral parts of this invention disclosure:

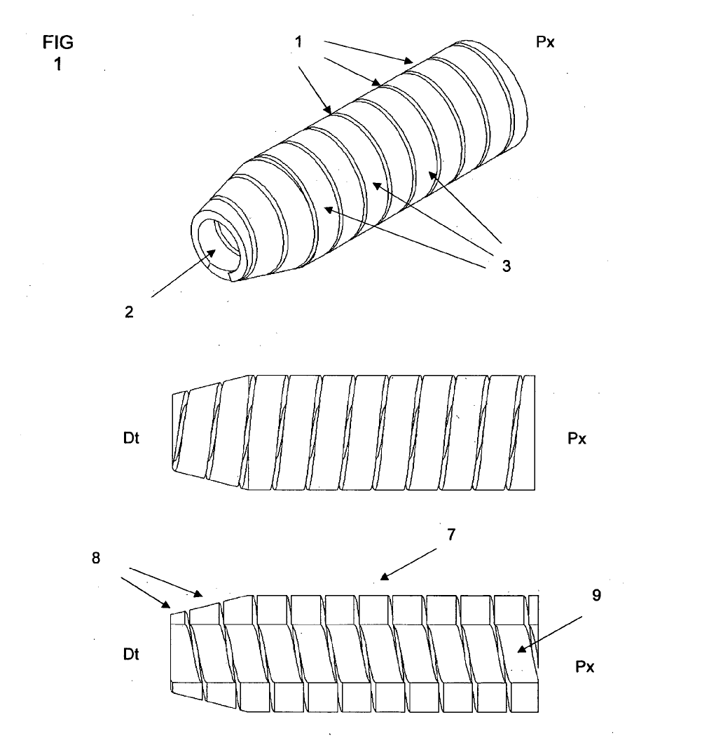

FIG. 1 shows a simple square spring rivet, with a single helix 1, an internal bore 2, and exterior surface 3, a proximal end Px, a distal end Dt, and a central area 7. The distal end has a lead taper 8, and the proximal end has a bore 9, which may receive a tool (not shown).

In this embodiment, the external and internal surfaces of the spring rivet are configured to have a substantially planar contact surface.

With regard to the internal contact surface, this enables a driver bit to have greater frictional contact with the connector than it would if the cross section of the wound length had been circular—as with a typical spring design.

Likewise, when the present invention is used with an aperture through a thicker sheet material, the planar contact surface provides an additional gripping surface.

A further advantage of having a planar contact outer surface is the avoidance of threading which can occur when the present invention is being used to join together two or more thin sheets. For example, a rounded external contact surface could effectively fit between two thin sheets when a connector is being wound therein. This could cause the sheets to be pushed apart as a result of the rivets ‘threads’ being wound between them.

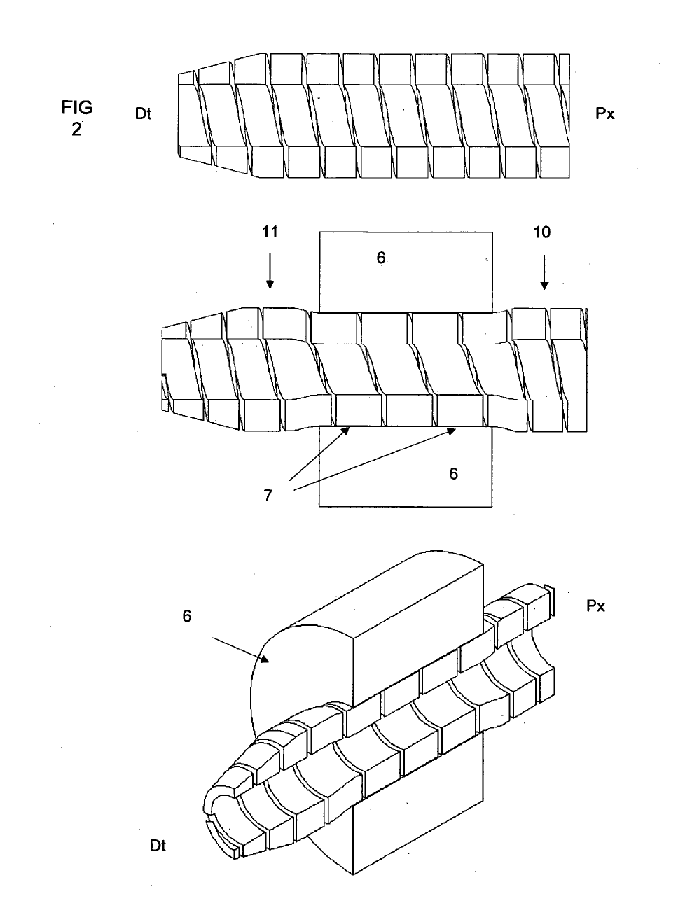

FIG. 2 shows a cross section the simple square spring rivet of FIG. 1 now inserted into the bore aperture 6. The central area 7 is reduced in diameter in the area of engagement with the bore aperture 6, but the proximal and distal ends are not reduced, retaining their original diameters, and therefore form respectively a head 10, and nut 11.

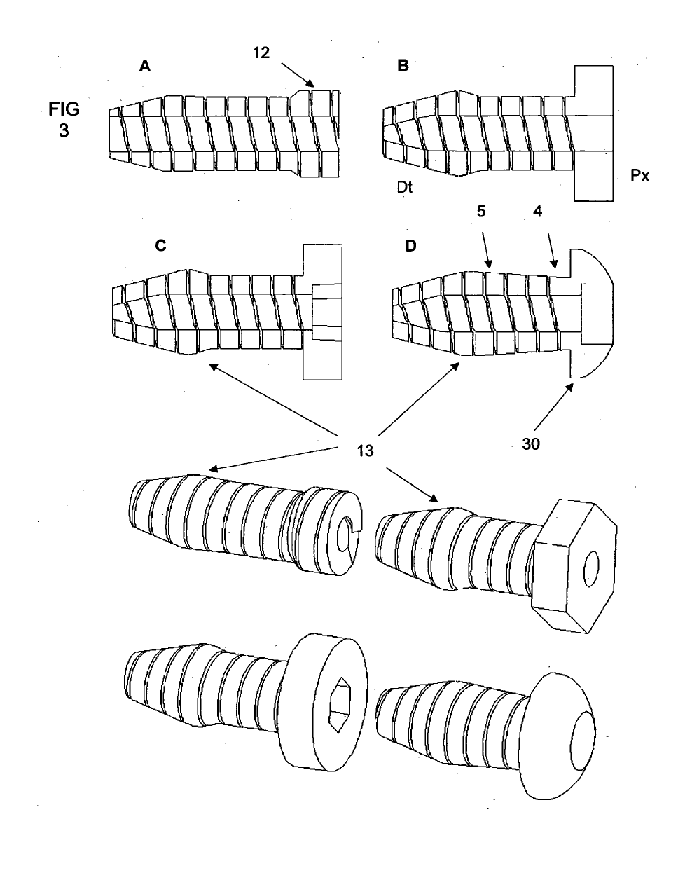

FIG. 3 shows cross sections and isometric views of four alternatives to the simple square spring rivet of FIG. 1, where there is a pre-formed head 12, and a pre-formed nut 13. FIGS. 3b 3c and 3d have more conventional solid form heads, but pre-formed smaller nut ends.

FIG. 3a. shows a form most similar to the original in FIG. 2, but with a preformed head and nut area. Therefore when inserted into a bore aperture there will be formed a larger diameter head and nut compared to the example in FIGS. 1 and 2. The pre-formed head is effectively a larger diameter area of the same helix of the main body.

FIG. 3b shows a tapered internal section and a hexagonal solid head.

FIG. 3c shows a reduced cross section at one end, and a hex cross-section hole which tapers inwards.

(Not shown here is where the internal bores and exterior surfaces vary in diameter, as indeed may the pitch of the helical cut or cuts.)

FIG. 3d shows a tapered internal section and a dome head. The outer surface of the fastener has a reverse tape aspect, being narrower 4 adjacent to the solid head, than in the area 5 of the pre-formed nut 30.

Note: The internal bores may be relatively large—as shown herein, for clarity—but may equally be very small, only just enough to allow for the compression for the spring fastener to be inserted.

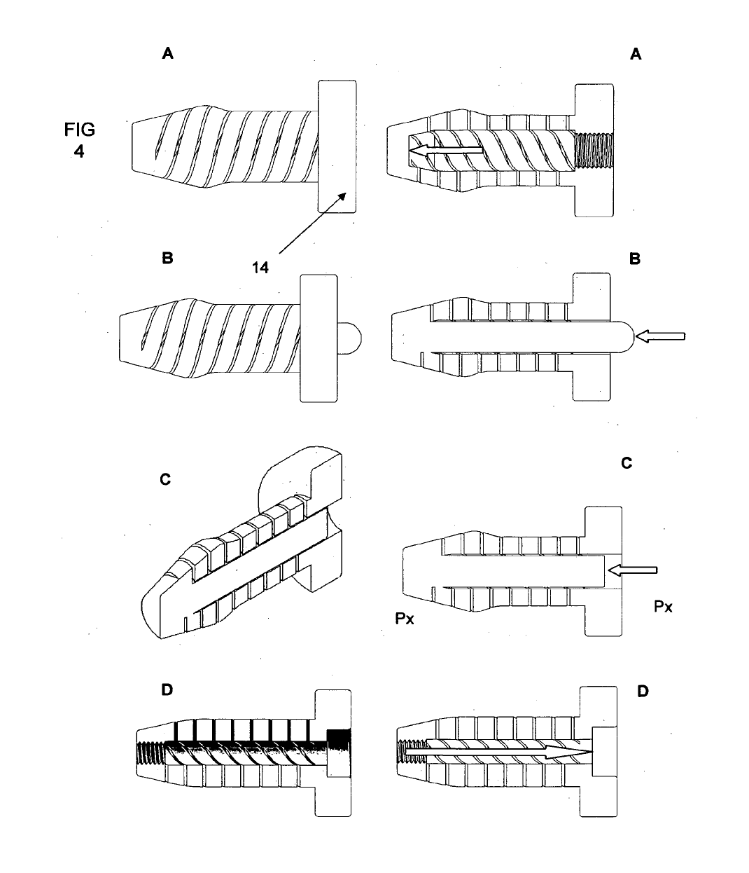

FIG. 4 shows a number of multi-start helix spring fastener where the head 14, at the proximal end Px, is in a solid form, where the head is attached to a central area.

FIG. 4a shows a spring fastener capable of being pre stretched by a threaded part (not shown) which pushes and/or turns in the direction of the arrow, on the distal end.

FIG. 4b shows a spring fastener which may be inserted by striking (in the direction of the arrow) a protruding internal pin element (shown here as a part of the spring fastener).

FIG. 4c shows a spring fastener similar to FIG. 4b, except the pin is recessed (and would be hit or pushed by an insertion pin as part of an insertion tool (these parts not shown).

Note: Alternatively to 4b and 4c the pin could be not connected to the spring fastener but be loose or indeed part of or attached to a mechanical, pneumatic, or hydraulic insertion tool.

FIG. 4d shows a spring fastener capable of compression after insertion to augment the self-locking force of the oversize nature of the spring fastener relative to the bore aperture. The compression element (not shown) could be an internal element that threads into the distal end of the spring fastener, and there can pull (or pull and turn) back to the proximal end (in the direction of the arrow), thereby shortening the spring fastener and increasing its outer diameter and lock to the bore aperture.

FIG. 5 shows a number alternative spring fastener forms

FIG. 5a shows a spring fastener made from a single continuous section of round section material. The proximal end 28 can be held and turned or pushed. there is no lead shown here but this could be fitted to a bore aperture which has a lead internally.

FIG. 5b shows a square section spring fastener with an external taper 27, and an internal taper 26. With detailing an internal drive pin element (not shown) could axial stretch the spring fastener, and then pass through the distal end (completely or partially), thereby allowing the spring fastener to retain its original shape—or as close as it can do so, given the restriction of the bore aperture it would fit within.

FIG. 5c shows a very simple spring fastener which may be fitted to a bore aperture with a lead. A tool may be frictionally engaged into the bore to use the spring fastener.

FIG. 5d shows a spring fastener with a taper and a solid head.

FIG. 6 shows a simple spring rivet, in its simplest form, shown from top to bottom views, in various stages of insertion (arrows 61) into a pair of plates 62. Insertion may be via frictional helical engagement of a tool (not shown) to the internal surface of the reduced internal diameter distal end 63. When inserted there is restriction at a middle area 64.

FIG. 7 shows a number of “helix” examples as per Definition 2, configured as simple spring rivets, simple spring rivet.

FIG. 7a is a single start simple spring rivet with a tip 76, a lead taper 77, a nut 78, a waist or grip 75, and a head 79.

FIG. 7b is a twin helix simple spring rivet. The figure shows the winds as separate parts for clarity, but they may be one wire, so that 70 and 71 could be joined/continuous wire, and or 72 and 73 could be joined/continuous wire.

FIG. 7c is a square section form of FIG. 7a which has an advantage in terms of surface area contact, or use with thin section of material for example metal house framing parts.

FIG. 7d is a simple spring rivet with a longer waist, 75, and a tension head 74, which winds back towards the tip end.

FIG. 7e is a simple spring rivet with an enlarged nut area.

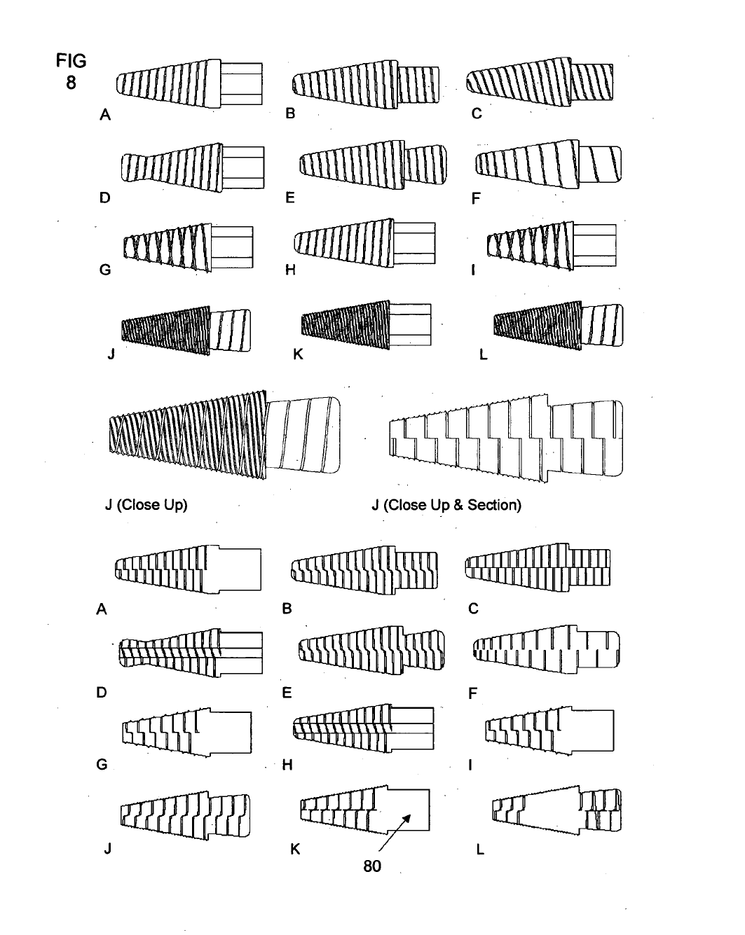

FIG. 8 shows a number of “helix” examples as per Definition 2, configured as an insert for dental restoration work.

FIG. 8a is a tapered helical insert, with a hexagonal post to which a dental crown may be attached.

FIG. 8b is a tapered helical insert, with a post to which a dental crown may be attached, where the post is circular and helical in form.

FIG. 8c is a multi-start helical form of FIG. 8b.

FIG. 8d is a tapered helical insert, with a post to which a dental crown may be attached, where the taper is modified to create a distal end which can secure into a pre formed dovetail detail in the bone.

FIG. 8e is a form of FIG. 8b where the post is dovetailed in form

FIG. 8f is a form of FIG. 8b where the helical pitch is shorter at one end.

FIG. 8g is a tapered helical insert, with a post to which a dental crown may be attached, with an internal bore, which may be plain as shown, or alternatively internal threaded. In either case an internal element may be inserted, or medication applied through the aperture.

FIG. 8h is a tapered helical insert, with a post to which a dental crown may be attached, where there is an external thread with a counter wind to the helical cut of the insert body.

FIG. 8i is a tapered helical insert, with a post to which a dental crown may be attached, where there is an external thread with a same direction wind to the helical cut of the insert body.

FIG. 8j is a tapered helical insert, with a helical post to which a dental crown may be attached, where there are multiple external threads with a counter wind to the helical cut of the insert body.

FIG. 8k is a tapered helical insert, with a solid post 80 to which a dental crown may be attached, where there are multiple external threads with a counter wind to the helical cut of the insert body.

FIG. 8l is a tapered helical insert, with a helical post to which a dental crown may be attached, and a helical tip end, where there is an external thread with a counter wind to the helical cut of the insert body, where a middle part is of the insert is solid in form.

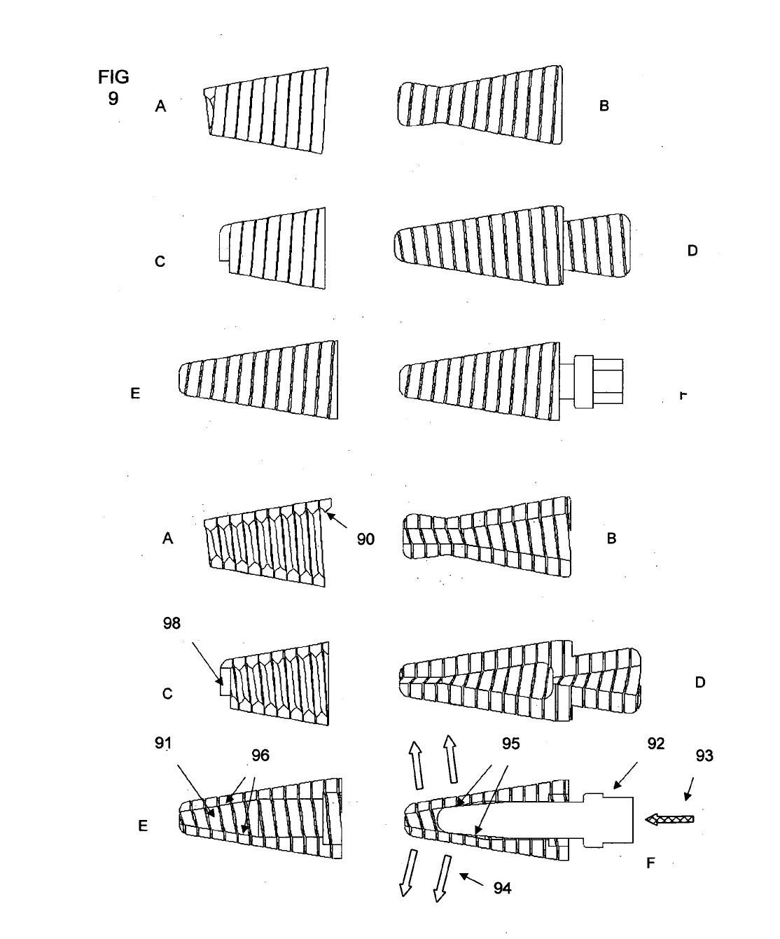

FIG. 9 shows a number of “helix” examples as per Definition 2, configured as an insert for dental restoration work, where the insert is configured for receiving a post (not shown except for in FIG. 9e).

FIG. 9a is a tapered helical insert, where the inside surface is configured as a thread.

FIG. 9b is a tapered helical insert, with a dovetail detail, and an internal bore.

FIG. 9c is a tapered helical insert, with a distal tang 98 which may be used to wind the insert in securely. Turning the tang will decrease the diameter of the helix as it is wound in.

FIG. 9d is a tapered helical insert, with a complex internal bore.

FIG. 9e is a tapered helical insert, for receiving a pin

FIG. 9f is the tapered helical insert, of FIG. 9e, showing a post pin 92, with a tapered distal end 95. In use the pin is inserted or wound into the insert in the direction of the arrow 93, which because the angle 95 of the pin is shallower than the angle 96 of the insert, the tip of the insert will form a dovetail detail against the jaw bone (which may be preformed to a suitable shape). The arrows 94 represent the dovetail expansion direction.

FIG. 10 shows three “helix” examples as per Definition 2, configured as a helical plug connector, for connection of kitset furniture. Whilst the examples here are double ended it should be appreciated that one end could have a hook or other detail from the prior art. In this way this embodiment could serve as fastener or a wall plug. The upper views shown are pre-insertion into a bore aperture, and the lower views (with arrows) are where the helical plug is now reduced in diameter and longer in length. (The bore aperture is not shown for clarity)

FIG. 10a shows a simple helical plug, which may be pushed and/or turned into a bore aperture.

FIG. 10b shows a helical plug, with an internal pin 103 which in use may be pushed and/or turned against a detail at the distal end 102 of the helical part. The pin has a shoulder 100 to avoid over insertion of the assembly into the bore aperture (not shown)

FIG. 10c shows a helical plug, with an internal pin 103 which may be pushed and or turned against the distal end 104 of the helical part. The pin has a shoulder 100 to avoid over insertion of the assembly into the bore aperture (not shown). In this example shown the pin and the associated bores of the helical parts are with sloping surfaces creating an anti-pullout dovetail feature.

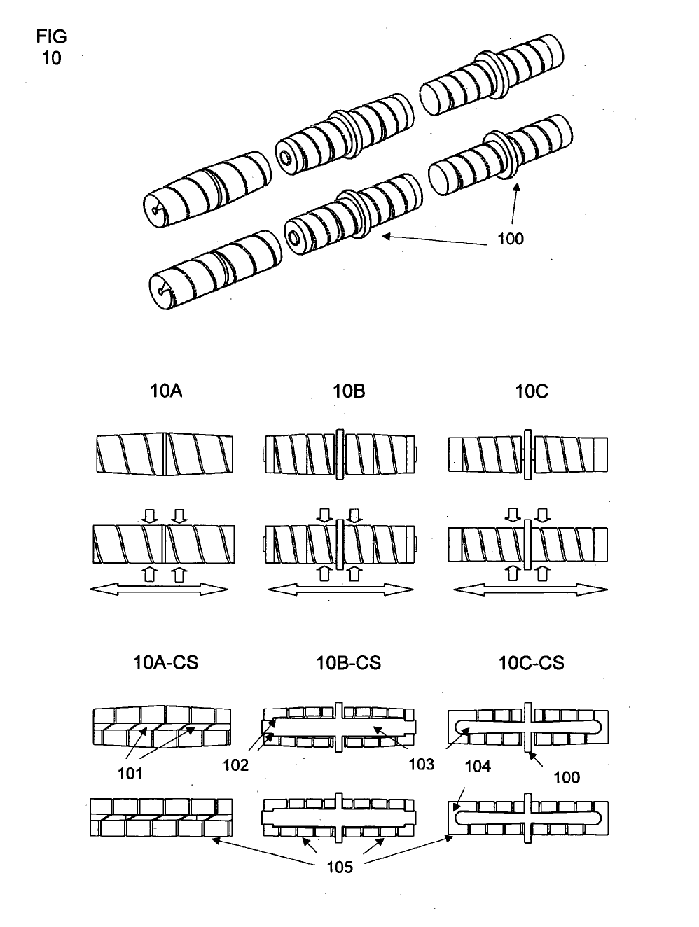

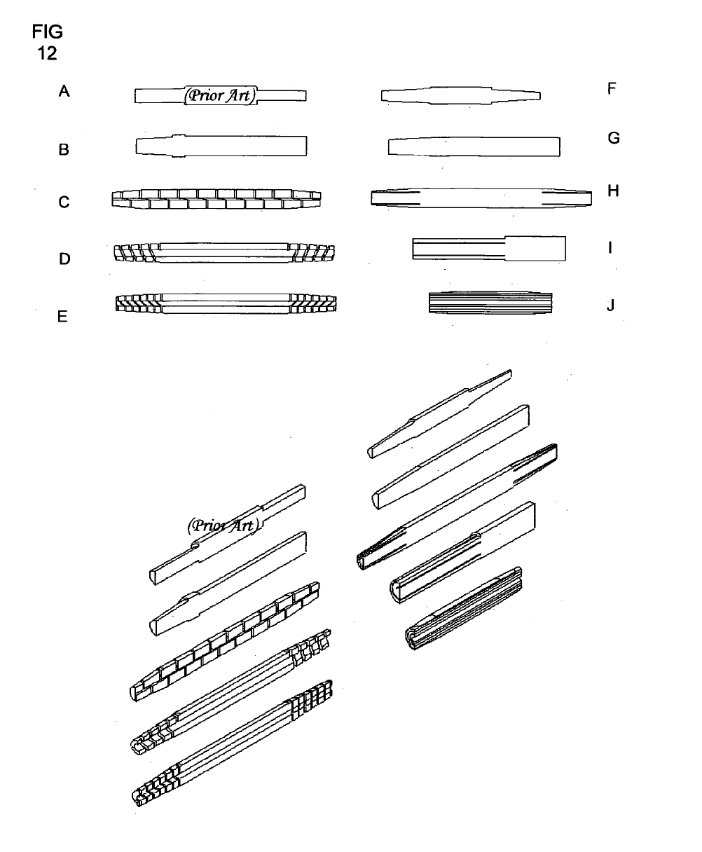

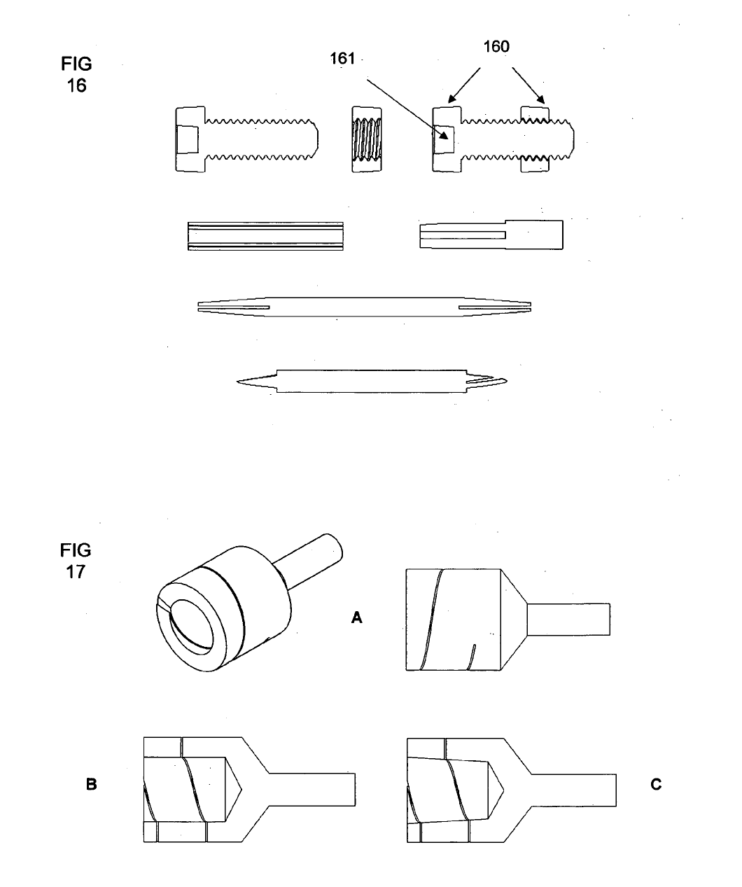

FIGS. 11-16 show a number of “helix” examples as per Definition 2, configured helical drive bit for use with a screw driver or power tool, to connect to a fastener, which may itself be helical but may equally be solid or conventional in form, other than it would have a generally circular aperture in the head for receiving the generally circular, maybe tapered drive bit. This invention describes both the fastener and the bit separately and in co-operation, used with any device such as a screw driver. The invention could also be applied to non fastener connection challenges.

FIGS. 11a, 12a, 13a are prior art for reference, but FIGS. 11b to 11j, 12b to 12j, 13c to 13e and 14h-14j show a number of alternative helical forms of this invention which may act as helical drive bits.

The driver bits may have same rotation or counter rotation detail of the two ends of a bit.

There may be a solid part as well as a helical part.

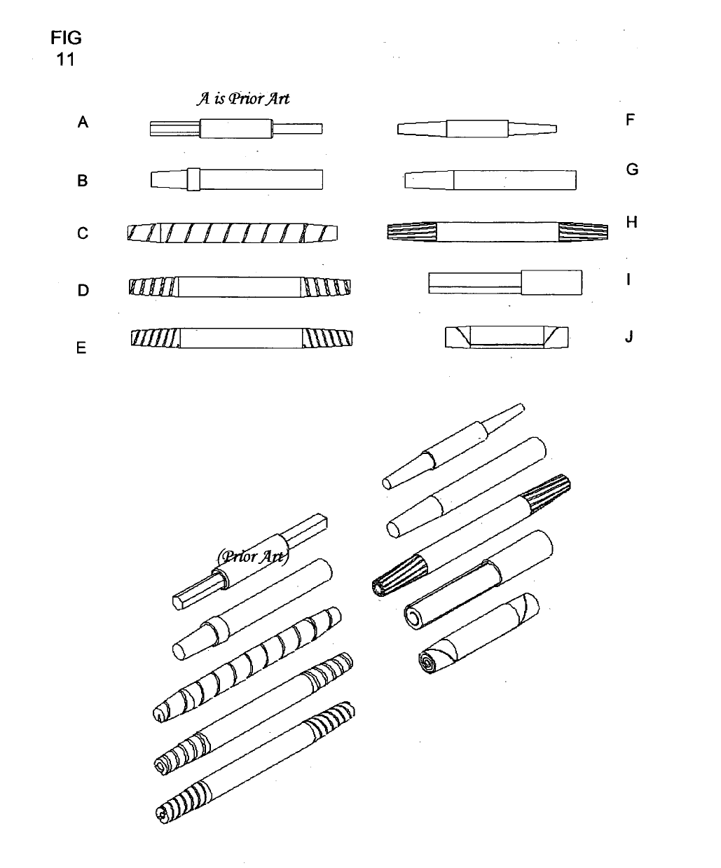

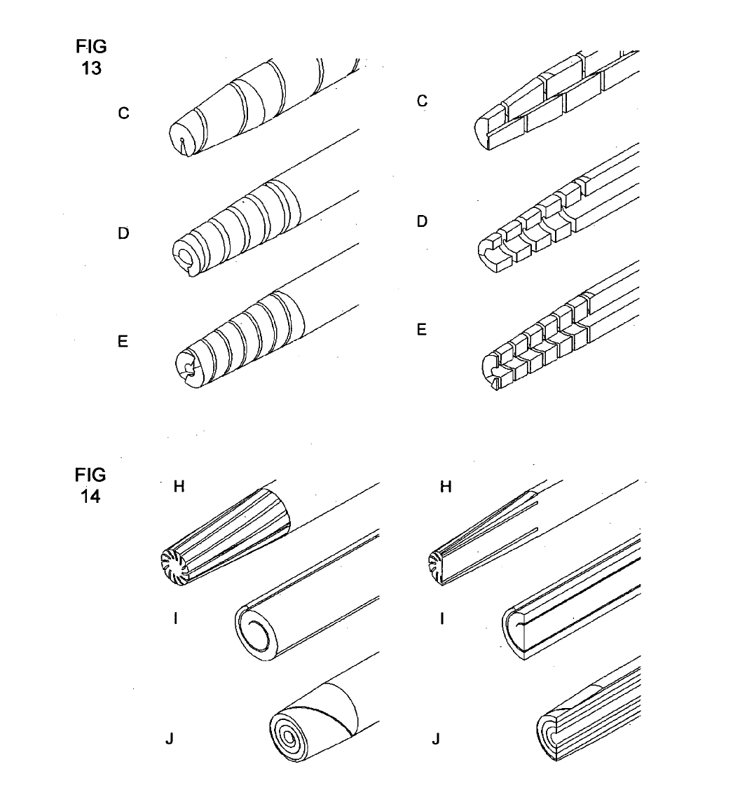

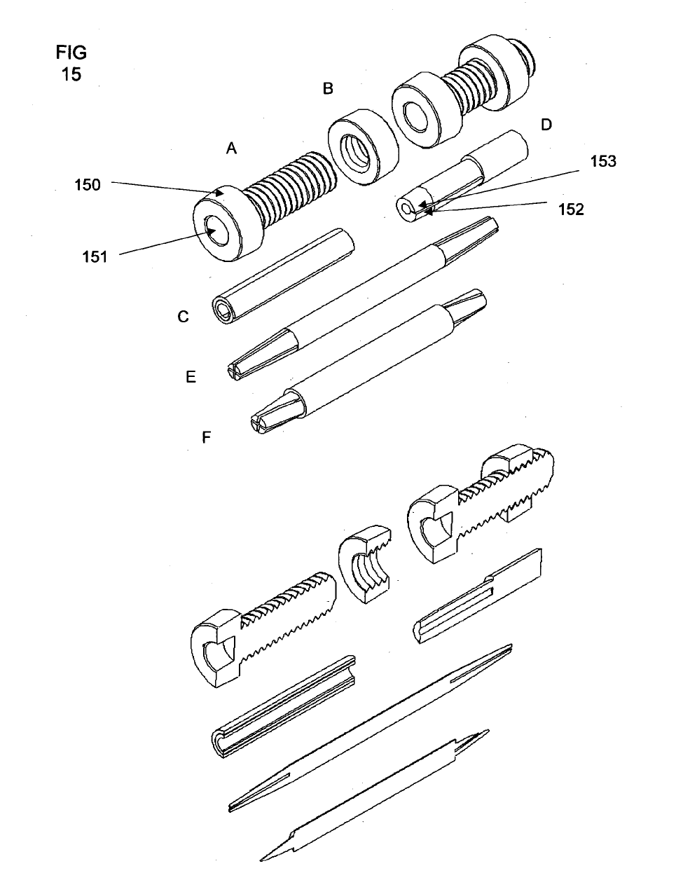

There may be one or more helical slots as in FIG. 12d, a wrap form as in FIG. 14j, an eccentric spiral form as in FIG. 14d, a twisted form as in FIG. 14h, or any other form or combination capable of interference fit and or helical fit to a bore aperture, which may be a nut as in the aperture 151 of FIG. 15a.

The nut and bolt in FIG. 15 show the versatility of this invention as the helical bit may be in male form, as shown herein, and fit to aperture 151, but it could equally be in female form (shown in Figure and fit to the surface 150 (which may be parallel splined or tapered).

The bolt in FIG. 15 could be used with either male or female form helical drive bits.

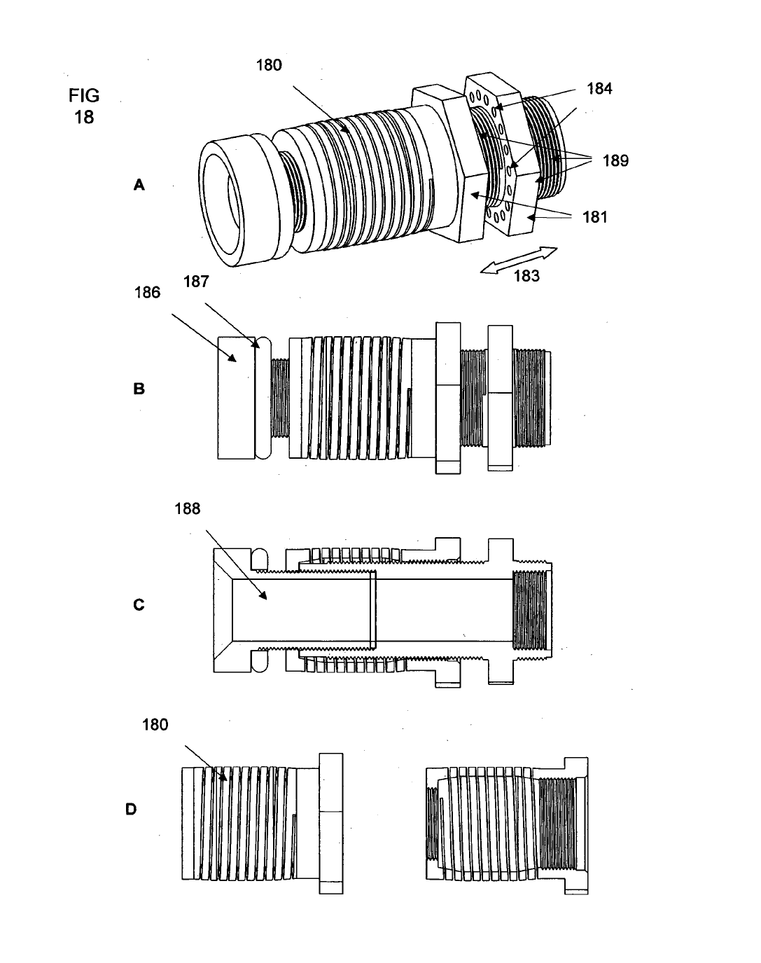

FIG. 17 show a number of “helix” examples as per Definition 2, configured as an external helical drive bit for use with power tool, to connect to the exterior generally circular cross section, fastener

FIG. 17a shows the general form of an exterior helical drive bit or connector.

FIG. 17b shows the general form of an exterior helical drive bit or connector, with a parallel internal bore.

FIG. 17c shows the general form of an exterior helical drive bit or connector, with a tapered internal bore.

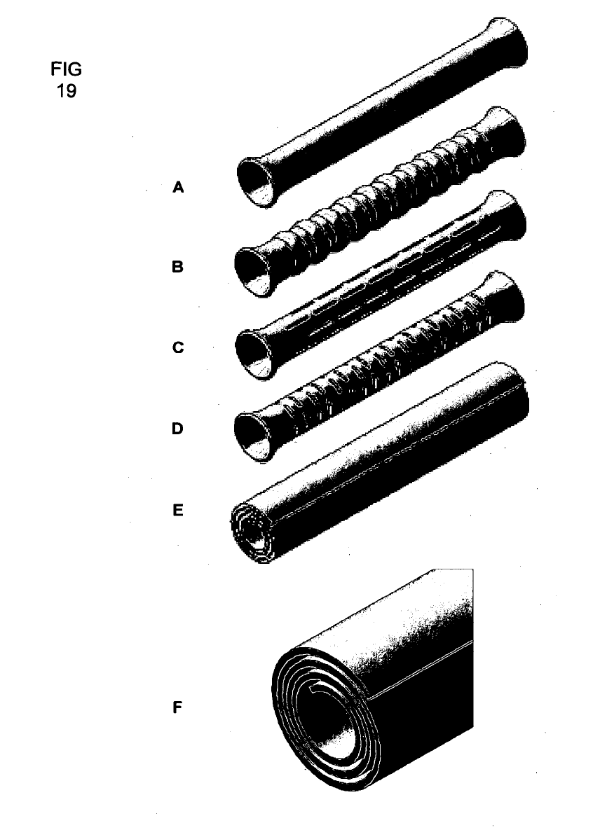

FIG. 18 shows a “helix” example as per Definition 2, with the general character that it may be either pre-tensioned helically (made longer and more slender) and then inserted into an “undersize” bore aperture, or alternately inserted into a bore aperture, and then post-tensioned to expand in a radial fashion in the internal bore of the bore aperture

FIG. 18a shows a helix assembly in an isometric view, with an outer helical part 180, and an inner tensioning part 189 which may apply linear and/or radial tension to the outer helical part 189, before and/or after insertion into a bore aperture (not shown). Tension may be applied via rotation, if threaded as shown, but may be via linear only. In either case the distance 183 between the details 181 will vary with the state of tension in the assembly.

FIG. 18b shows a side view of the assembly of FIG. 18a

FIG. 18c shows a cross section side view of the assembly of FIG. 18a.

Because the helical elements can be slender, there can remain a substantial internal void 188 for the flow of liquid of gas, as in the case of the leaking Gulf of Mexico oil well.

FIG. 18d shows the helical part of the assembly by itself.

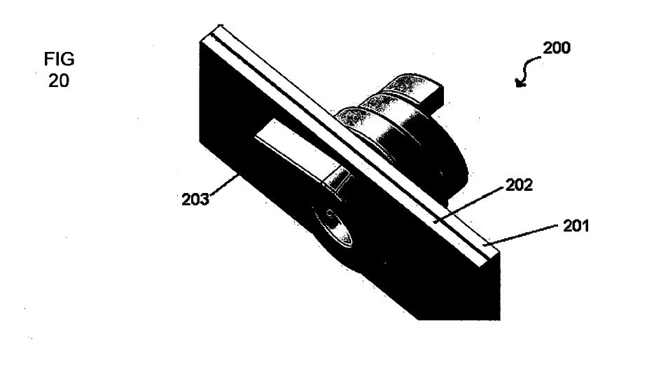

FIG. 19 shows a number of “helix” examples as per Definition 2, with the general character that they are able to expand in a radial fashion in the internal bore dimension and then subsequently via a reaction force frictionally lock on an inserted wire or rod (not shown).

FIG. 19a shows a tube which may be made from an elastomer material and therefore able to lock frictionally to an adjacent bore aperture.

FIG. 19b shows a tube ribbed in character and therefore able to lock frictionally to an adjacent bore aperture.

FIG. 19c shows a tube slotted in character and therefore able to lock frictionally to an adjacent bore aperture.

FIG. 19d shows a tube alternatively slotted in character and therefore able to lock frictionally to an adjacent bore aperture.

FIG. 19e shows a tube which is a spiral in character, and therefore able to lock frictionally to an adjacent bore aperture.

FIG. 19f shows a close up of the end of the spiral form illustrated in FIG. 19e.

FIG. 20 illustrates a connector in the form of a spring fastener generally indicated by arrow (200). The spring fastener (200) has been used in this embodiment to hold together two sheets of material (201 and 202).

In this embodiment, the spring fastener (200) includes a head in the form of a tang (203). The tang (203) is essentially a section of the spring fastener (200) which extends outside of the spiral of the body of the fastener (200).

Aspects of the present invention have been described by way of example only and it should be appreciated that modifications and additions may be made thereto without departing from the scope thereof as defined in the appended claims.

What we claim is:

1. A connector for use in an aperture, comprising:
a body having a helix which has at least one portion with an outer diameter greater than at least part of the inner diameter of the aperture; and wherein
at least part of the body is hollow in an axial direction; and
the helix is configured to change circumferentially in response to a change in force applied to the connector such that the portion of the helix having a greater outer diameter than the aperture at least partially passes through the aperture, wherein the portion extends outwards after the portion or parts of the portion passes through the aperture.

2. A connector as claimed in claim 1 wherein the portion of the helix with a greater outer diameter than the aperture is configured to aid extraction of the connector.

3. A connector as claimed in claim 1 wherein the helix is made of elastic material with a material memory.

4. A connector as claimed in claim 1 wherein the portion of the helix having a greater diameter than the aperture is configured to return to its original structure upon passing through the aperture in order to act as a nut.

5. A connector as claimed in claim 1 wherein the connector includes a head.

6. A connector as claimed in claim 5 wherein the head is in the form of a tang.

7. A connector as claimed in claim 1 wherein one end of the body is in the form of a drill.

8. A connector as claimed in claim 1 wherein the body is configured to carry a tool.

9. A connector as claimed in claim 8 wherein the helix is configured such that a tool can be attached to it.

10. A connector as claimed in claim 1 wherein the force is applied in the form of rotational torque.

11. A connector as claimed in claim 1 wherein the force is applied in the form of linear force.

12. A connector as claimed in claim 1 wherein the helix has at least one portion with a tapered outer circumference.

13. A connector as claimed in claim 1 wherein the helix has at least one portion with a tapered inner circumference.

14. A connector as claimed in claim 1 wherein the body is in the form of an unbroken wound spiral.

15. A connector as claimed in claim 1 wherein the external and internal surfaces of the helix on the body are configured to have a substantially planar contact surface.

16. A fastening system including a connector as claimed in claim 10 and a driver bit, wherein the driver bit includes
a body having an attachment portion at one end, wherein the attachment portion is configured to be connected to a rotational driver, and wherein
the body having, at the distal end to the attachment portion, a shaft configured to engage with the internal circumference of the connector.

17. A fastening system as claimed in claim 16 which includes components with pre-drilled apertures for the insertion of the connector.

18. A method of fastening a connector, as claimed in claim 1, to a component having an aperture, the method comprising the steps of:
a) inserting the connector into the aperture;
b) applying rotational torque via a driver bit to the connector to change the circumference of the portion of the helix having a greater diameter than the aperture;
c) further inserting the portion of the helix of the body into the aperture;
d) releasing the driver bit to change the circumference of the body such that the portion of the helix having a greater diameter than the aperture expands outwardly after the portion, or parts of the portion passes through the aperture.

19. A method as claimed in claim 18 wherein the aperture is the bone of an animal.

20. A method as claimed in claim 19 wherein the bone is a jaw bone.
